(19) United States
(12) Patent Application Publication
(10) Pub. No.: US 2018/0064778 A1
(43) Pub. Date: Mar. 8, 2018

(54) Compositions and Methods for Improvement of Mood

(71) Applicant: IN Ingredients, Inc., Columbia, TN (US)

(72) Inventors: Bolin Qin, Gaithersrburg, MD (US); Augustin T. Romero, Columbia, TN (US); Tim Romero, Columbia, TN (US)

(21) Appl. No.: 15/693,665

(22) Filed: Sep. 1, 2017

Related U.S. Application Data
(60) Provisional application No. 62/382,933, filed on Sep. 2, 2016.

(51) Int. Cl. A61K 36/53; A61K 36/88; A61K 31/56

(52) U.S. Cl. CPC A61K 36/53; A61K 31/56; A61K 36/88

(57) ABSTRACT

Processes are provided for altering an expression of a mood condition or disorder in a subject without a sleep disorder includes administering to the subject without a sleep disorder in need thereof an effective amount of an extract of rosemary, an extract of Hemerocallis fulva, an active component thereof, or combinations thereof at an administration time.

### CROSS-REFERENCE TO RELATED APPLICATIONS

This application claims priority to U.S. Provisional Patent Application Ser. No. 62/382,933, filed Sep. 2, 2016, and entitled “Compositions and Methods for Improvement of Mood,” the entirety of which is incorporated by reference herein.

### FIELD

Various embodiments described herein relate to compositions and methods for the improvement of mood in a subject.

### BACKGROUND

With mounting pressures of work, family life, finances, relationships and other factors, there is an ever increasing feeling of negativity that pervades many members of society. It is common to express feelings of depression, lack of energy, negative thoughts, among other mood related issues. Often such feelings are linked to a lack of sleep or sleep quality in the subject. However, such is not always the case. For those individuals, medicaments that assist in sleep are of little value. As such there is a need for processes and compositions for mood enhancement without the need for sleep adjustment.

### SUMMARY

In various embodiments, a process for altering a mood condition in a subject without a sleep disorder includes administering to the subject without a sleep disorder in need thereof an effective amount of an extract of rosemary, an extract of Hemerocallis fulva, an active component thereof, or combinations thereof at an administration time. The administration time is optionally daily once or more times, and optionally for a period of one week or more, optionally two weeks or more. Such administration has been shown to improve a condition of mood such as lack of enthusiasm or depression in subjects.

### DETAILED DESCRIPTION

The following description of particular aspect(s) is merely exemplary in nature and is in no way intended to limit the scope of the invention, its application, or uses, which may, of course, vary. Various embodiments are described with relation to the non-limiting definitions and terminology included herein. These definitions and terminology are not designed to function as a limitation on the scope or practice of the invention but are presented for illustrative and descriptive purposes only. While the processes or compositions are described as an order of individual steps or using specific materials, it is appreciated that steps or materials may be interchangeable such that the description of the embodiments may include multiple parts or steps arranged in many ways as is readily appreciated by one of skill in the art.

It is appreciated herein that the singular forms “a,” “an,” and “the” include plural reference unless the context clearly dictates otherwise. The term “including” is used to mean “including but not limited to.”

Provided are processes for treating, preventing, or modulating a mood or a mood related disorder without modulating sleep parameters in a subject. Mood is optionally one or more of altered feelings of tiredness, attentiveness, alertness, grogginess, focus, sluggishness, energy, lethargy, enthusiasm, depression, fatigue, restedness, among others. Methods for measuring such values are known in the art, optionally through the use of the Leeds Sleep Evaluation Questionnaire (Parrott, A C and Hindmarch, I, Psychopharmacology (Berl). 1980; 71(2):173-9) alone or combined with other questionnaires for one or more of the above subjective measurements.

As used herein, a subject is defined as an organism (such as a human, non-human primate, equine, bovine, murine, or other mammal), or a cell. Illustrative examples of cells include neuronal cells, optionally sympathetic neuronal cells, optionally sensory neuronal cells.

In some aspects, negative feelings of mood are referred to herein as a disease or condition (e.g. disorder). A process includes administering to a subject having a disease or condition associated with an undesirable mood and in need of such treatment an effective amount of a composition that is or includes an extract of rosemary, an extract of Hemerocallis fulva, active portion thereof, or combinations thereof or a dietary supplement containing in whole or in part of an extract of rosemary, an extract of Hemerocallis fulva, active portion thereof, or combinations thereof. Such an administration is at an administration time. The administration of the extract of rosemary, an extract of Hemerocallis fulva, active portion thereof, or combinations thereof or a dietary supplement containing such will alter an a mood condition or disorder. As such, the invention has utility for treating, preventing, or otherwise modulating a mood condition.

In some aspects, the disease or condition is independent of a sleep disorder, optionally insomnia, jet lag, shift work sleep disorder, delayed sleep phase syndrome (DSPS), advanced sleep phase syndrome (ASPS), a non-24-hour sleep wake disorder or irregular sleep-wake pattern. In some aspects, treating comprises ameliorating symptoms of the disease or condition.

Provided are materials that are optionally in the form of botanical extracts, such as an extract of rosemary, an extract of Hemerocallis fulva, active portion thereof, or combinations thereof, alone or as part of a dietary supplement that have utility for altering one or more characteristics of a mood condition or disorder in a subject. The extract, active portion thereof, synthetic form thereof, or dietary supplement may be in pharmaceutical dietary supplement composition in solid, semi-solid, or liquid dosage forms, such as, for example, tablets, chewables, suppositories, pills, capsules, powders, liquids, or suspensions, and may be provided in unit dosages suitable for a single administration. Time release preparations are also contemplated as effective dosage formulations. The compositions may include an effective amount of a selected extract of rosemary, extract of Hemerocallis fulva, active portion thereof, or combinations, optionally in combination with a pharmaceutically acceptable carrier and, in addition, may include or exclude other medicinal agents, pharmaceutical agents, carriers, or diluents.

The compositions are recognized to include an active ingredient alone or optionally as an active portion of an extract, or the sole portion or sole active portion of an extract. “Active ingredient” refers a component present in the extract that renders, directly or indirectly, the intended effect of the extract. In some aspects, an active ingredient is ursolic acid. Extraction parameters such as water quality, heating temperature, drying temperature, heating time, drying time, and filtering processes all contribute to the quality and efficiency of the process. Water quality directly affects the concentration of active ingredient(s). Poor quality water may cause active ingredient(s) to become decomposed or oxidized during or following the extraction process.

In some aspects, rosemary or Hemerocallis fulva may be obtained from various resources. Rosemary, or Rosmarinus officinalis, is a woody bush native to the Mediterranean region. Extracts of rosemary may be made from Rosmarinus spp. and preferably from the leaves or young flowering tops of fresh rosemary (Rosmarinus officinalis L. and its cultivars). Rosemary extraction may be performed by harvesting the leaves of a rosemary plant and reducing them in size such as by chopping to improve solvent penetration. A typical particle size is optionally 0.5-5.0 mm, or any value or range therebetween. In some aspects, the leaf is chopped into a powder type substance with a particle size of less than 0.5 mm. The chopped plant material is combined with a suitable extraction solvent such as water and/or a low molecular weight alcohol (e.g. C₄-C₆ alcohol) such as ethanol. The plant material is combined with the solvent for an extraction time of 18 to 36 hours. The extraction temperature is optionally the range 10° C. to 45° C. The resulting extract liquid is separated from the solid material and filtered, optionally with a sterile filter. Optionally, the resulting extract is poured onto nonstick tray and allowed to dry at 80-90° C. Vacuum-spray dry equipment is optionally used for the drying procedure. The resulting dry extract powder is weighed. An extraction ratio is calculated as w/20×100% with w as the weight (g) of the dry extract powder. The sample and water ratio, heat time, volume of water in the second extraction may vary depending on the amount of the raw material used for extraction.

Extracts of Hemerocallis fulva are optionally obtained from extraction in an extraction solvent such as water and/or low molecular weight alcohol such as ethanol. Extracts are prepared from plants belonging to the genus Hemerocallis of the family Liliaceae of the order Liliales are used. Examples of such plants include Akinowasuregusa (Hemerocallis fulva L. sempervirens M. Hotta or Hemerocallis sempervirens Araki), Honkanzo (Hemerocallis fulva L. var. fulva), Nokanzo (Hemerocallis fulva L. var. longituba Maxim or Hemerocallis longituba Miq.), and Yabukanzo (Hemerocallis fulva L. var. kwanso Regal). An extract is optionally prepared from whole plants or plant parts, such as flower buds, leaves, stems, or roots.

In some aspects, an extract is prepared by drying the plant material and optionally cutting the material into a suitable size for extraction, such as the sizes described for rosemary. The plant material is combined with an extraction solvent (e.g. water, aqueous buffer, low molecular weight alcohol, or combinations thereof) that is preheated to a temperature of 60° C. to 100° C. for an extraction time, typically of 20 to 90 minutes. The particulate material is then removed by gravity separation, centrifugation, or filtering, optionally with a filter size suitable for aseptic filtration. The resulting extract is optionally poured onto nonstick tray and allowed to dry at 80-90° C. Vacuum-spray dry equipment is optionally used for the drying procedure. The resulting dry extract powder is weighed. An extraction ratio is calculated as w/20×100% with w as the weight (g) of the dry extract powder. The sample and water ratio, heat time, volume of water in the extraction may vary depending on the amount of the raw material used for extraction.

In some aspects, a second extraction of either plant material is performed optionally using the same extraction parameters or differing extraction parameters. Optionally, a second extraction is performed in a low molecular weight alcohol optionally of C₂-C₄. The first and second extraction solutions are optionally combined together and dried.

In some aspects, a composition includes or consists of ursolic acid as an active ingredient. Ursolic acid is optionally present in a composition at a concentration of 10 weight percent or greater. In some aspects, ursolic acid is present in an extract at a concentration of 25 weight percent or greater. Optionally, ursolic acid is present at a weight percent at or in excess of 5, 10, 15, 20, 25, 30, 35, 40, 45, 50, 55, 60, 65, 70, 75, 80, 85, 90, 95, 96, 97, 98, 99, 99.1, 99.2, 99.3, 99.4, 99.5, 99.6, 99.7, 99.8, or 99.9.

While ursolic acid is descried as a component of or the product representing an extract, it is appreciated that the term “extract” may in some aspects include otherwise isolated, purified, or chemically synthesized ursolic acid. As such, an “extract” is in some aspects ursolic acid obtained either by extraction from a natural or non-natural source, or used as a chemically synthesized ursolic acid.

Depending on the intended mode of administration, the composition can be in pharmaceutical compositions in the form of solid, semi-solid or liquid dosage forms, such as, for example, tablets, suppositories, pills, capsules, powders, liquids, or suspensions, and may be provided in unit dosages suitable for a single administration. Time release preparations are specifically contemplated as effective dosage formulations. The compositions will include an effective amount of the selected extract in combination with a pharmaceutically acceptable carrier and, in addition, may include other medicinal agents, pharmaceutical agents, carriers, or diluents.

In a solid composition aspect, conventional nontoxic solid carriers may include, for example, pharmaceutical grades of mannitol, lactose, starch, magnesium stearate, sodium saccharine, talc, cellulose, glucose, sucrose and magnesium carbonate. Liquid pharmaceutically administrable compositions may, for example, be prepared by dissolving or dispersing an active compound with optimal pharmaceutical adjuvants in an excipient, such as water, saline, aqueous dextrose, glycerol, or ethanol, to form a solution or suspension. For example, the pharmaceutical composition may contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art; for example, see Remington's The Science and Practice of Pharmacy (20^{th} Edition).

In oral administration aspects, fine powders or granules of extract, or a liquid extract may contain diluting, dispersing, or surface active agents. The extract may be presented in water or in syrup, in capsules or sachets in the dry state, or in a non-aqueous solution or suspension. Suspending agents may also be included in tablets, which may include binders and lubricants in a suspension. Flavoring, preserving, suspending, thickening, or emulsifying agents may be also included to modify the taste and texture of the composition. The tablets and granules provided for oral administration may further be coated for ease of digestion.

In some aspects, the extract containing dietary supplement composition may be combined with one or more other active agents. An active agent optionally functions synergistically with an extract material. Active agents illustratively include vitamins (such as vitamin A, vitamin B, vitamin C, vitamin D, vitamin E or vitamin K), antioxidants (such as acai, wolfberry, alpha lipoic acid, astazanthin, or fucoxanthin), or any combination of the above. The extract according to the present invention is available as a food additive thereto. Examples include foods in a liquid, semi-liquid, solid, paste, or jelly form.

A process optionally includes administration of an active component optionally of an extract or dietary supplement containing extract at an administration time in an amount effective to treat the disease or condition or otherwise modulate or ameliorate a symptom of such a mood disease of condition. Administration is optionally once daily, twice daily or more. Administration optionally is done 1, 2, 3, 4 or more times each day. Optionally, administration is done on an as needed basis. Optionally irregularly, or weekly. Optionally, administration is done once or twice weekly. Optionally, an administration time is following exercise. Optionally, an administration time when one or more symptoms of a disease or condition are present, or conditions exist that such a symptom is expected or may occur. Optionally, administration is prior to, following, during, or in lieu of a meal, snack or other consumption of food or nutritious drink. Optionally, administration is prior to, during or following the consumption of alcohol. Optionally, an administration time is at the initiation of a work period.

Administration time is optionally from −4 hours to 12 hours following light exposure. Optionally, administration is from 0 to 12 hours after light exposure. Optionally, an administration time is upon waking independent of the time of day or the onset of light exposure. Optionally, an administration time is in the evening. In some aspects, the extract is administered in a form that releases at certain times, optionally in an extended release form, in a periodic release form, or using a time control pump.

In a typical regimen, the extract materials are taken orally between one and three times daily; although, other routes of administration may be utilized. Also, it should be noted that the extracts of the present invention may be utilized in the form of derivatives. For example, the extracts may be bonded, chemically or physically, to other species and moieties such as synthetic polymers, liposomes, small organic molecules, chitin, chitosan, other biopolymers and the like. In view of the teaching presented herein, still further combinations will be readily apparent to those of skill in the art.

A subject is administered a composition in a dosage so that each dose of the extract supplement selected to deliver an amount of active agent suitable to have an effect on mood. Variable dosing regimens are operative. While in some instances, a single dose treatment may be effective in producing therapeutic effects, in other instances a treatment period in the range of, for example, six weeks to three or six months or more may be utilized. The composition may be administered orally, parentally, or intravenously, intramuscularly, intraperitoneally, by transdermal injection, or by contact with a cell or tissue such as by immersion or other form of contact. Injectables may be prepared in conventional forms, either liquid solutions or suspensions, solid forms suitable for solution or prior to injection, or as suspension in liquid prior to injection or as emulsions.

The composition may be provided in sterile form and contain an effective amount of one or more agents for producing the desired response in a unit of weight or volume suitable for administration to a subject. The doses of extracts or dietary supplements containing gone or more of the extracts are administered to a subject can be chosen in accordance with different parameters in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication.

The dose of the composition may vary depending on the age, weight, general condition of the subject. For example, dosage is in the range of 1-1,000 mg of equivalent of dry extract powder extract per day may be an effective range. In some aspects, a dosage is between 10 and 800 mg per day, optionally from 100 to 600 mg per day, optionally 200-400 mg per day, optionally 400 mg per day. Dosage is optionally 1, 2, 3, 4, or more times daily. Optionally, dosage is twice daily. Optionally, dosage is 200 mg twice daily. Optionally, dosage is 500 mg twice daily. The extract may also comprise 0.01%-100% of the dry weight of the composition. For example, a dietary supplement composition may comprise 20%-50% of the dry weight of the extract composition. An “effective amount” is defined as that capable of altering one or more characteristics of a mood condition relative to a control.

An extract optionally is or is a part of a dietary supplement composition. An extract is optionally present in a dietary supplement composition at 10%-100% by weight, optionally 20%-50% by weight, optionally 30%-40% by weight, or any value or range between 10% and 100% of the dry weight of the dietary supplement composition.

Various aspects of the present invention are illustrated by the following non-limiting examples. The examples are for illustrative purposes and are not a limitation on any practice of the present invention. It will be understood that variations and modifications can be made without departing from the spirit and scope of the invention.

### EXAMPLES

A total of 32 healthy men and women were enrolled in the study. For subject selection, in addition to a sub-clinical score on the Insomnia Severity Index, other factors used for screening included BMI (subjects could not have a BMI of greater than 40 kg/m², since morbid obesity is known to negatively impact sleep quality (Tishler P. V., Larkein E. K., Schluchter M. D., Redline S. Incidence of sleep-disordered breathing in an urban adult population: the relative importance of risk factors in the development of sleep-disordered breathing. JAMA. 2003, 289(17), 2230-2237)); use of dietary supplements (subjects could not be using dietary supplements designed to improve sleep quality or dietary supplements designed to increase energy); use of medications for purposes of improving sleep quality (including prescription medications and off-the-shelf medications); use of other sleep aids (subjects could not be using other types of sleep aids); smoking habits (subjects needed to be non-smokers); and use of alcohol (subjects could not be consuming alcohol beverages at a quantity of greater than 3 drinks per week). In addition, women could not be pregnant or nursing. A health history, medication and dietary supplement usage, and physical activity questionnaire was completed by all subjects to determine eligibility. Women were required to take a urine pregnancy test to confirm that they were not pregnant. Prior to participation, each subject was informed of procedures, potential risks, and benefits associated with the study, and subjects provided written informed consent prior to being admitted to participate.

Subjects were equally and randomly assigned in a double-blind manner to one of two conditions: (1) placebo (rice bran powder: one 500 mg capsule nightly for weeks 1-2 and two capsules nightly for weeks 5-6), or (2) supplement (one 500 mg capsule nightly for weeks 1-2 and two capsules nightly for weeks 5-6). IN-Ingredients (Columbia, Tenn.) provided the supplement, containing a 1:1 by weight blend of rosemary (Rosmarinus officinalis) and Hemerocallis fulva. The supplement contained ursolic acid at not less than 10% by weight. Both the placebo and supplement capsules were produced by a contract manufacturer and were of near identical appearance.

Capsules were distributed to subjects in unlabeled bottles, with a known quantity of capsules in each bottle. A total of 16 subjects were assigned to each group. The each group included 6 men and 10 women. Women subjects started the protocol during the initial week of their menstrual cycle to control for potential changes in estradiol and other variables across the cycle. Although both men and women were enrolled in the study, there were no attempts to compare the results between the genders.

Subjects were instructed to ingest the capsule(s) one hour prior to bedtime, ingest the capsule(s) at least two hours following their last meal, and turn in the bottle at the end of week 2. The same bottle was returned to the subjects at the start of week 5. A two-week washout period was included during weeks 3 and 4, in which the subjects did not consume any capsules. Capsule counting upon bottle return was used to determine compliance of intake.

Subjects reported to the lab on four different days over the course of the six week period: at the baseline, and at the end of weeks 2, 4, and 6. The time of day for lab reporting was standardized for each subject across the days of testing and occurred during the early morning hours (e.g., between 6 am and 8 am). The following data was collected: heart rate (using an automated unit); blood pressure (using an automated unit); respiratory rate (via counting for a 60 second period); blood sample (approximately 7 ml of blood was collected via venipuncture); questionnaire related to mood and energy level; and open ended subject comments pertaining to their sleep quality. For the questionnaire related to mood and energy level, a 10-point scale was used where 0 was “none” and 10 was “extreme,” and subjects indicated the degree to which they “felt” each descriptor. Regarding the open ended subject comments, subjects were asked to describe their overall quality of sleep over the past two weeks, including items such as ease of falling asleep, frequency of waking at night, tossing and turning during the night, ease of arising in the morning, and energy level upon rising.

In addition to the laboratory measures, the Leeds Sleep Evaluation Questionnaire (Parrott A. C., Hindmarch, I. The Leeds Sleep Evaluation Questionnaire in psychopharmacological investigations—a review. Psychopharmacology (Berl). 1980; 71(2): 173-9) was completed on the morning of days 4, 5, 6, and 7 or weeks 2, 4, and 6. Subjects did not record sleep duration or naptime throughout the course of the study. Additionally, although other tools are available for assessing sleep quality aside from the Leeds Questionnaire, it should be understood that sleep quality was only one outcome related to this study.

Subjects were instructed to consume their usual diet throughout the study period and to record all food and drink consumed during the three days prior to each test day. Diet records were analyzed for nutrient intake using computer software, specifically, Food Processor Pro available from Esha Research (Salem, Oreg.). Subjects were instructed not to consume alcohol after 7:00 pm or during the 48 hours prior to each test day. Additionally, subjects were instructed to not consume caffeine after 2:00 pm each day. Subjects were further instructed to continue with their usual program of physical activity throughout the study period and were asked to make no changes in their sleep conditions throughout the study period, including a change in mattress, pillow, bedding, sounds machine, or other supportive sleep aids. No data was collected related to physical activity or sleep conditions.

The data were analyzed using a condition×time repeated measures analysis of variance, using all time points of data collection in the overall model. Accordingly, effects of a single or double dosage of the assigned capsule was considered. Effect sizes were calculated using Cohen's d. The data are presented as mean±SE. Statistical significance was set at P<0.05.

All thirty-two subjects successfully completed the protocol. Compliance to capsule intake for subjects assigned to the supplement group was 96% and for subjects assigned to the placebo group was 98%, with no statistically significant difference noted (p>0.05). No adverse outcomes were noted with treatment, and all subjects tolerated the treatment well. However, one subject reported feelings of weakness, one subject reported abdominal discomfort, one subject reported increased irritability, and eight subjects reported lethargy, with seven of these being in the supplement group.

Subject characteristics are presented in Table 1, with no statistically significant differences noted between conditions (p>0.05). Caffeine intake was higher (p=0.03) for subjects in the placebo group (244±63 mg) compared to subjects in the supplement group (102±16 mg).

TABLE 1
Characteristics of men and women assigned to a supplement
(containing rosemary and Hemerocallis fulva) or a placebo

| | Supplement | | Placebo | |
|---|---|---|---|---|
| Variable | (N = 16) | | (N = 16) | |
| Age (years) | 30.6 ± | 2.4 | 26.9 ± | 1.9 |
| Height (cm) | 172.4 ± | 2.6 | 172.3 ± | 3.0 |
| Weight (kg) | 75.2 ± | 4.3 | 73.4 ± | 4.4 |
| BMI (kg*m⁻²) | 25.0 ± | 0.92 | 24.5 ± | 1.1 |
| Waist (cm) | 81.3 ± | 2.5 | 78.6 ± | 3.5 |
| Hip (cm) | 97.0 ± | 1.9 | 94.3 ± | 2.4 |
| Waist: Hip | 0.84 ± | 0.01 | 0.83 ± | 0.02 |
| Heart Rate (bpm) | 75.2 ± | 4.5 | 70.3 ± | 2.5 |
| Systolic Blood Pressure (mmHg) | 122.6 ± | 2.1 | 121.9 ± | 2.8 |
| Diastolic Blood Pressure (mmHg) | 77.4 ± | 2.2 | 76.0 ± | 2.1 |
| Years anaerobic exercise training | 4.9 ± | 1.7 | 1.4 ± | 0.43 |
| Hours per week anaerobic exercise | 1.7 ± | 0.55 | 1.5 ± | 0.42 |
| Years aerobic exercise training | 4.9 ± | 1.3 | 4.8 ± | 2.2 |
| Hours per week aerobic exercise | 2.4 ± | 0.45 | 2.1 ± | 0.35 |
| Insomnia Index | 15.9 ± | 1.3 | 15.6 ± | 1.4 |

Data are mean ± SE
No differences of statistical significance were noted for any variable (p > 0.05).

Data for heart rate, blood pressure, and respiratory rate are presented in Table 2. No interaction effects were noted, nor were there any differences between conditions or across time for any measure (p>0.05).

TABLE 2
Heart rate, blood pressure, and respiratory rate of men and
women assigned to a supplement (containing rosemary and
Hemerocallis fulva) or a placebo

| | | | | |
|---|---|---|---|---|
| Variable | Baseline | Week 2 | Week 4 | Week 6 |

Heart Rate (bpm)

| | | | | | | | | |
|---|---|---|---|---|---|---|---|---|
| Supple- | 69.8 ± | 2.8 | 70.5 ± | 2.9 | 72.3 ± | 3.3 | 71.8 ± | 3.1 |
| ment | | | | | | | | |
| Placebo | 73.2 ± | 3.4 | 75.0 ± | 3.0 | 72.0 ± | 2.8 | 69.9 ± | 2.5 |

Systolic Blood Pressure (mmHg)

| | | | | | | | | |
|---|---|---|---|---|---|---|---|---|
| Supple- | 120.7 ± | 2.8 | 115.9 ± | 1.7 | 116.7 ± | 3.5 | 118.4 ± | 2.2 |
| ment | | | | | | | | |
| Placebo | 121.5 ± | 3.0 | 123.1 ± | 3.0 | 119.3 ± | 2.8 | 120.3 ± | 3.5 |

Diastolic Blood Pressure (mmHg)

| | | | | | | | | |
|---|---|---|---|---|---|---|---|---|
| Supple- | 75.4 ± | 2.5 | 73.9 ± | 2.3 | 76.3 ± | 2.9 | 72.7 ± | 2.2 |
| ment | | | | | | | | |
| Placebo | 75.7 ± | 2.7 | 76.1 ± | 2.9 | 76.9 ± | 2.9 | 74.6 ± | 3.0 |

Respiratory Rate (breaths/min)

| | | | | | | | | |
|---|---|---|---|---|---|---|---|---|
| Supple- | 17.5 ± | 0.33 | 17.8 ± | 0.33 | 17.6 ± | 0.18 | 17.5 ± | 0.24 |
| ment | | | | | | | | |
| Placebo | 18.4 ± | 0.52 | 17.9 ± | 0.27 | 17.9 ± | 0.15 | 17.8 ± | 0.17 |

Values are mean ± SE.
No differences of statistical significance were noted for any variable (p > 0.05).
Note:
Data was collected at Baseline (before beginning supplement or placebo), and at the end of weeks 2, 4, and 6. During weeks 3 and 4, subjects did not ingest any supplement or placebo capsules.

At a dosage level of one capsule per day, subjective mood and energy levels were not impacted by treatment across time in a statistically significant way, as can be seen in Table 3 (p>0.05). However, a condition effect was noted for alertness (p=0.04), with values higher for the supplement group compared to the placebo group. Time effects were noted for tiredness (p=0.001), sluggishness (p=0.05), energetic (p=0.02), well-rested (p=0.0002), and fatigued (p=0.002). In all cases, small improvements were noted across time in the above variables for the supplement group, with beneficial changes noted at week 2 and week 6, as shown in Table 3.

TABLE 3
Subjective feelings of men and women assigned to a supplement
(containing rosemary and Hemerocallis fulva) or a placebo

| | | | | |
|---|---|---|---|---|
| Variable | Baseline | Week 2 | Week 4 | Week 6 |

Attentiveness

| | | | | |
|---|---|---|---|---|
| Supplement | 6.4 ± 0.39 | 6.7 ± 0.41 | 6.3 ± 0.39 | 6.8 ± 0.34 |
| Placebo | 6.0 ± 0.39 | 6.6 ± 0.26 | 6.5 ± 0.30 | 5.9 ± 0.53 |

Tiredness*

| | | | | |
|---|---|---|---|---|
| Supplement | 6.4 ± 0.45 | 5.1 ± 0.48 | 5.4 ± 0.56 | 5.4 ± 0.56 |
| Placebo | 6.4 ± 0.39 | 4.4 ± 0.31 | 5.1 ± 0.41 | 4.1 ± 0.43 |

Alertness**

| | | | | |
|---|---|---|---|---|
| Supplement | 6.8 ± 0.36 | 6.6 ± 0.33 | 6.1 ± 0.49 | 6.8 ± 0.31 |
| Placebo | 5.6 ± 0.36 | 6.0 ± 0.39 | 6.4 ± 0.27 | 6.1 ± 0.49 |

Groggy

| | | | | |
|---|---|---|---|---|
| Supplement | 4.8 ± 0.55 | 4.1 ± 0.56 | 4.2 ± 0.55 | 4.1 ± 0.52 |
| Placebo | 5.4 ± 0.54 | 3.7 ± 0.44 | 3.9 ± 0.57 | 3.5 ± 0.61 |

Focused

| | | | | |
|---|---|---|---|---|
| Supplement | 6.1 ± 0.36 | 6.5 ± 0.42 | 5.8 ± 0.53 | 6.5 ± 0.47 |
| Placebo | 5.6 ± 0.35 | 6.3 ± 0.40 | 6.2 ± 0.37 | 6.0 ± 0.26 |

Sluggishness*

| | | | | |
|---|---|---|---|---|
| Supplement | 4.8 ± 0.57 | 4.0 ± 0.51 | 4.4 ± 0.57 | 3.9 ± 0.55 |
| Placebo | 5.4 ± 0.55 | 3.5 ± 0.42 | 3.6 ± 0.53 | 3.8 ± 0.60 |

Energetic*

| | | | | |
|---|---|---|---|---|
| Supplement | 5.4 ± 0.44 | 5.6 ± 0.41 | 5.6 ± 0.36 | 6.4 ± 0.32 |
| Placebo | 4.7 ± 0.34 | 6.2 ± 0.43 | 5.8 ± 0.35 | 6.0 ± 0.41 |

Lethargic

| | | | | |
|---|---|---|---|---|
| Supplement | 4.1 ± 0.63 | 3.9 ± 0.56 | 3.3 ± 0.50 | 3.4 ± 0.41 |
| Placebo | 4.4 ± 0.50 | 3.4 ± 0.46 | 3.3 ± 0.50 | 3.1 ± 0.54 |

Enthusiasm

| | | | | |
|---|---|---|---|---|
| Supplement | 6.4 ± 0.41 | 6.2 ± 0.42 | 5.8 ± 0.45 | 6.5 ± 0.42 |
| Placebo | 5.8 ± 0.32 | 6.8 ± 0.32 | 6.5 ± 0.30 | 5.9 ± 0.42 |

Depressed

| | | | | |
|---|---|---|---|---|
| Supplement | 2.1 ± 0.45 | 2.1 ± 0.50 | 2.4 ± 0.57 | 2.0 ± 0.47 |
| Placebo | 2.8 ± 0.58 | 2.3 ± 0.58 | 2.3 ± 0.52 | 2.3 ± 0.62 |

Well-Rested*

| | | | | |
|---|---|---|---|---|
| Supplement | 4.4 ± 0.40 | 6.0 ± 0.52 | 4.8 ± 0.32 | 5.9 ± 0.50 |
| Placebo | 3.7 ± 0.34 | 5.6 ± 0.53 | 5.6 ± 0.48 | 6.1 ± 0.51 |

Fatigued*

| | | | | |
|---|---|---|---|---|
| Supplement | 5.1 ± 0.47 | 3.9 ± 0.55 | 3.8 ± 0.45 | 2.8 ± 0.59 |
| Placebo | 5.5 ± 0.48 | 3.2 ± 0.47 | 4.4 ± 0.44 | 3.5 ± 0.52 |

Values are mean ± SE.
*Time effect noted; improvements were noted across time in these variables for the supplement group.
**Condition effect noted for alertness; supplement > placebo (p > 0.04).
Note:
Data was collected at Baseline (prior to beginning supplement or placebo) and at the end of weeks 2, 4, and 6. During weeks 3 and 4, subjects did not ingest any supplement or placebo capsules.

During weeks 5 and 6 subjects were assigned double dosage. It was noted that at this higher dosage, subjects assigned to the supplement reported an 8% increase in attentiveness, an 11% increase in alertness, a 12% increase in focus, a 14% increase in a feeling of being energetic, a 12% increase in enthusiasm, a 23% increase in feeling well-rested, an 11% decrease in feelings of sluggishness, and a 16% decrease in feeling depressed (all percent change values relative to week 4 values). These absolute values were not statistically different across time, but are presented to provide an overview of the degree of change noted with treatment. Subjects assigned to the placebo experienced small or no positive changes in regards to the above variables. The corresponding effect size for the above variables is as follows: attentiveness (d=0.34); alertness (d=0.43); focused (d=0.35); energetic (d=0.59); enthusiasm (d=0.40); well-rested (d=0.67); sluggishness (d=0.22); and depressed (d=0.19).

Although no findings of statistical significance were noted for sleep quality and no condition×time interaction effects were noted for any outcome measure, certain subjective measures appear to be improved for subjects assigned to the supplement group, in particular, while ingesting two capsules per day.

Various modifications of the present invention, in addition to those shown and described herein, will be apparent to those skilled in the art of the above description.

Embodiments can be described with reference to the following numbered clauses, with preferred features laid out in the dependent clauses:

1. A process of improving a condition of mood in a subject, optionally a human subject, without a sleep disorder comprising administering to said subject without a sleep disorder and in need thereof a composition comprising an effective amount of an extract of rosemary, an extract of Hemerocallis fulva, an active component thereof, or combinations thereof at an administration time, the administration for a period of one week or more.

2. The process of clause 1, wherein said extract is a water extract.

3. The process of clause 1 or 2, wherein extract is present at a dosage of 500 mg or greater.

4. The process of any preceding clause, wherein said administering is once daily, optionally twice daily.

5. The process of any preceding clause, wherein said administration is from −4 hours to 12 hours or 0 to 12 hours after light exposure.

6. The process of any of clauses 1-4, wherein said administration is from 1-3 hours after light exposure or 10-14 hours after light exposure.

7. The process of any of clauses 1-4, wherein said administration is from 4 hours to 0 hours prior to light exposure.

8. The process of any preceding clause, wherein said composition comprises an extract of Hemerocallis fulva.

9. The process of any preceding clause, wherein said composition comprises an extract of Hemerocallis fulva and an extract of rosemary.

10. The process of any preceding clause, wherein said extract comprises ursolic acid at 10% by weight or greater.

11. The process of any preceding clause, wherein said extract comprises ursolic acid at 25% by weight or greater.

12. The process of any preceding clause, wherein said extract consists of ursolic acid.

13. The process of any preceding clause, wherein said administration is for a period of 2 weeks or more.

14. The process of any preceding clause, wherein said administration is for four weeks or more.

15. The process of any preceding clause, wherein said administration is for three months or more.

16. The process of any preceding clause, wherein said administration is once, twice, three, or four times daily.

17. The process of any preceding clause, wherein said composition is in the form of a powder, gel, liquid, food, solid, or other form.

18. The process of any proceeding clause wherein the composition comprises or consists of an extract of rosemary and an extract of Hemerocallis fulva.

19. The process of clause 18 wherein the extract of rosemary and an extract of Hemerocallis fulva are present at about a 1:1 ratio by weight.

20. The process of clauses 18 or 19 wherein the composition comprises 10% by weight or more ursolic acid.

21. The process of clause 20 wherein the composition comprises 25% by weight or more ursolic acid.

22. The process of clauses 18, 19, 21, or 21 wherein the administration is twice daily.

23. The process of clause 22 wherein the composition is present at 500 mg per dose or greater.

It is appreciated that all reagents are obtainable by sources known in the art unless otherwise specified.

Patents, publications, and applications mentioned in the specification are indicative of the levels of those skilled in the art to which the various embodiments pertain. These patents, publications, and applications are incorporated herein by reference to the same extent as if each individual patent, publication, or application was specifically and individually incorporated herein by reference.

The foregoing description is illustrative of particular embodiments, but is not meant to be a limitation upon the practice thereof.

1. A process for improving a mood condition in a human subject without a sleep disorder comprising:
administering to said subject in need thereof and without a sleep disorder an effective amount of a composition comprising an extract of rosemary, an extract of Hemerocallis fulva, or combinations thereof at an administration time;
said administration at least one time daily for a period of one week or more.

2. The process of claim 1, wherein said extract is a water extract.

3. The process of claim 1, wherein extract is administered at a dose of 500 milligrams or more.

4. The process of claim 1, wherein said administering is twice daily.

5. The process of claim 1, wherein said step of administering is from −4 hours to 12 hours or 0 to 12 hours after light exposure.

6. The process of claim 1, wherein said step of administering is from 1-3 hours after light exposure or 10-14 hours after light exposure.

7. The process of claim 1, wherein said step of administering is from 4 hours to 0 hours prior to light exposure.

8. The process of claim 1, wherein said extract is an extract of Hemerocallis fulva.

9. The process of claim 1, wherein said extract is an extract of Hemerocallis fulva and rosemary present at about a 1:1 ratio by weight.

10. The process of claim 1, wherein said extract comprises ursolic acid at 10% by weight or greater.

11. The process of claim 1, wherein said extract comprises ursolic acid at 25% by weight or greater.

12. The process of claim 1, wherein said extract consists of ursolic acid.

13. The process of claim 1, wherein said step of administering is for a period of four weeks or more.

14. The process of claim 1, wherein said step of administering is for six weeks or more.

15. The process of claim 1, wherein said step of administering is for three months or more.

16. The process of claim 1, wherein said step of administering is three or four times daily.

17. The process of claim 1, wherein said composition is in the form of a powder, gel, liquid, food, solid, or other form.

18. The process of claim 1 wherein the condition is lack of enthusiasm or depression.
